(19) United States
(12) Patent Application Publication
(10) Pub. No.: US 2004/0029161 A1
(43) Pub. Date: Feb. 12, 2004

(54) Methods for genomic analysis

(75) Inventors: David R. Cox, Belmont, CA (US); Nila Patil, Woodside, CA (US); Anthony J. Berno, San Jose, CA (US); David A. Hinds, Mountain View, CA (US)

Correspondence Address: PERLEGEN SCIENCES, INC., LEGAL DEPARTMENT, 2021 STIERLIN COURT, MOUNTAIN VIEW, CA, 94043, US

(73) Assignee: Perlegen Sciences, Inc., Mountain View, CA

(21) Appl. No.: 10/448,773

(22) Filed: May 30, 2003

Related U.S. Application Data
(60) Provisional application No. 60/421,137, filed on May 30, 2002.

(51) Int. Cl. G06F 17/60; C12Q 1/68; G06F 19/00; G01N 33/48; G01N 33/50

(52) U.S. Cl. 435/6; 702/20; 705/3

(57) ABSTRACT

The present invention relates to business methods for discovery of therapeutic and diagnostic products by identifying variations that occur in the human genome, relating these variations to one another, and, ultimately, relating these variations to the genetic bases of phenotype such as disease resistance, disease susceptibility or drug response.

### CROSS-REFERENCE TO RELATED APPLICATIONS

The present application claims priority to U.S. provisional patent application serial No. 60/421,137, filed May 30, 2002 entitled “Methods for Genomic Analysis” to U.S. provisional patent application serial No. 60/313,264 filed Aug. 17, 2001, to U.S. provisional patent application serial No. 60/327,006, filed Oct. 5, 2001, all entitled “Identifying Human SNP Haplotypes, Informative SNPs and Uses Thereof”, provisional patent application serial No. 60/337/567 filed Nov. 30, 2001 entitled “Methods for Identifying Evolutionary Conserved Sequences”, and U.S. utility patent application Ser. No. 10/106,097, filed Mar. 26, 2002, entitled “Methods for Genomic Analysis”, the disclosures all of which are specifically incorporated herein by reference in their entirety and for all purposes.

### BACKGROUND OF THE INVENTION

The DNA that makes up human chromosomes provides the instructions that direct the production of all proteins in the body. These proteins carry out the vital functions of life. Variations in the sequence of DNA encoding a protein produce variations or mutations in the proteins encoded, possibly affecting the normal function of cells. Although environment often plays a significant role in disease, variations or mutations in the DNA of an individual are directly related to almost all human diseases, including infectious disease, cancer, and autoimmune disorders.

Because any two humans are 99.9% similar in their genetic makeup, most of the sequence of the DNA of their genomes is identical. However, there are variations in DNA sequence between individuals. For example, there are deletions of many-base stretches of DNA, insertion of stretches of DNA, variations in the number of repetitive DNA elements in non-coding regions, and changes in single nitrogenous base positions in the genome called “single nucleotide polymorphisms” (SNPs). Human DNA sequence variation accounts for a large fraction of observed differences between individuals, including susceptibility to disease.

Although most SNPs are rare, it has been estimated that there are 5.3 million common SNPs, each with a frequency of 10-50%, that account for the bulk of the DNA sequence difference between humans. Such SNPs are present in the human genome once every 600 base pairs (Kruglyak and Nickerson, Nature Genet. 27:235 (2001)). Alleles (variants) making up blocks of such SNPs in close physical proximity are often “linked” or correlated, such that these variants do not recombine independently. This results in reduced genetic variability and defines a limited number of “SNP haplotypes”, each of which reflects descent from a single, ancient ancestral chromosome (Fullerton, et al., Am. J. Hum. Genet. 67:881 (2000)).

The complexity of local SNP haplotype structure in the human genome—and the distance over which individual haplotypes extend—is poorly defined. Empiric studies investigating different segments of the human genome in different populations have revealed tremendous variability in local haplotype structure. These studies indicate that the relative contributions of mutation, recombination, selection, population history and stochastic events to haplotype structure vary in an unpredictable manner, resulting in some haplotypes that extend for only a few kilobases (kb), and others that extend for greater than 100 kb (A. G. Clark et al., Am. J. Hum. Genet. 63:595 (1998)).

Any comprehensive description of the haplotype structure of the human genome, defined by variants or SNPs, will require empirical analysis of a dense set of SNPs in many independent copies of the human genome. Thus, methods for analyzing data to determine the variant or SNP haplotype structure of the genome are of great interest in the art.

### SUMMARY OF THE INVENTION

The present invention relates to business methods for identifying variations that occur in the human genome, relating these variations to one another, and, ultimately, relating these variations to the genetic bases of phenotype such as disease resistance, disease susceptibility or drug response. In one embodiment, the methods allow, once variants have been identified, determination of variant haplotype blocks and patterns, and further, comparing the frequencies of SNP haplotype patterns from coding regions of individuals from a control group with the frequencies of SNP haplotype patterns from coding regions of individuals from a case group, to identify genetic loci related to a phenotype present in the case group but not in the control group, and further to use the identified genetic loci for discovery and development of therapeutic and diagnostic products.

Specifically, the present invention provides a method for determining disease-related genetic loci without a priori knowledge of a sequence or location of said disease-related genetic loci, comprising determining SNP haplotype patterns using a group of SNPs consisting essentially of common SNPs from individuals in a control population; determining SNP haplotype patterns using a group of SNPs consisting essentially of common SNPs from individuals in a diseased population; and comparing frequencies of the SNP haplotype patterns of the control population with frequencies of the SNP haplotype patterns of the diseased population, wherein differences in the frequencies indicate locations of disease-related genetic loci.

In addition, the present invention provides a method for determining an informative SNP in a SNP haplotype pattern, comprising: from a group consisting essentially of common SNPs, determining SNP haplotype patterns for a SNP haplotype block; comparing each SNP haplotype pattern of interest in the SNP haplotype block to the other SNP haplotype patterns of interest in that SNP haplotype block; selecting at least one SNP in the first SNP haplotype pattern of interest that distinguishes that first SNP haplotype pattern of interest from other SNP haplotype patterns of interest in the SNP haplotype block, wherein the selected SNP is an informative SNP for the first SNP haplotype pattern.

Also, the present invention provides a method for selecting a SNP haplotype block useful in genomic analysis, comprising: isolating substantially identical DNA strands from about five different individuals for analysis; analyzing at least 1×10⁶ bases from each substantially identical DNA strand; determining SNP locations in each DNA strand; discarding SNP locations that occur in less than a pre-determined frequency of the strands; identifying undiscarded SNP locations in the DNA strands that are linked to other undiscarded SNP locations, wherein said linked SNP locations form a SNP haplotype block; identifying SNP haplotype patterns that occur in each SNP haplotype block; and selecting each identified SNP haplotype pattern.

### BRIEF DESCRIPTION OF THE FIGURES

The following figures and drawings form part of the present specification and are included to further demonstrate certain aspects of the patent invention.

FIG. 1 is a schematic of one embodiment of the methods of the present invention from identifying variant locations to associating variants with phenotype to using the associations to identify drug discovery targets or as diagnostic markers.

FIG. 2 shows sample SNP haplotype blocks and SNP haplotype patterns according to the present invention.

FIG. 3 is a schematic showing one embodiment of a method for selecting SNP haplotype blocks.

FIG. 4 illustrates a simple employment of one embodiment of the method shown in FIG. 3.

FIG. 5A is a schematic of one embodiment of a method for choosing a final set of SNP haplotype blocks. FIG. 5B is a simple employment of the method shown in FIG. 5A. The “letter:number” designations in FIG. 5B indicate “haplotype block ID:informativeness value” for each block.

FIG. 6 is a schematic of one embodiment of using the methods of the present invention in an association study.

FIG. 7 shows the haplotype patterns for twenty independent globally diverse chromosomes defined by 147 common human chromosome 21 SNPs.

The present invention relates to methods for identifying variations that occur in the human genome, relating these variations to one another, and relating these variations to the genetic bases of disease and drug response. In particular, the present invention relates to resolving ambiguous or missing data in the process of identifying individual variations or SNPs, determining a data set of SNP haplotype sequences, and determining a pattern set of SNP haplotype patterns.

### DETAILED DESCRIPTION OF THE INVENTION

It readily should be apparent to one skilled in the art that various embodiments and modifications may be made to the invention disclosed in this application without departing from the scope and spirit of the invention. All publications mentioned are cited for the purpose of describing and disclosing reagents, methodologies and concepts that may be used in connection with the present invention; nothing herein should be construed as an admission that these references are prior art in relation to the inventions described herein.

As used in the specification, “a” or “an” means one or more. As used in the claim(s), when used in conjunction with the word “comprising”, the words “a” or “an” mean one or more. As used herein, “another” means at least a second or more.

As used herein, “individual” refers to a specific single organism, such as a single animal, human insect, bacterium, etc.

As used herein, “informativeness” of a SNP haplotype block is defined as the degree to which a SNP haplotype block provides information about genetic regions.

As used herein, the term “informative SNP” refers to a genetic variant such as a SNP or subset of SNPs (more than one) that tends to distinguish one SNP haplotype pattern from other SNP haplotype patterns within a SNP haplotype block.

As used herein, the term “isolate SNP block” refers to a SNP haplotype block that consists of one SNP.

As used herein, the term “linkage disequilibrium”, “linked” or “LD” refers to genetic loci that tend to be transmitted from generation to generation together; e.g., genetic loci that are inherited non-randomly.

As used herein, the term “singleton SNP haplotype” or “singleton SNP” refers to a specific SNP allele or variant that occurs in less than a certain portion of the population.

As used herein, the term “SNP” or “single nucleotide polymorphism” refers to a genetic variation between individuals; e.g., a single nitrogenous base position in the DNA of organisms that is variable. As used herein, “SNPs” is the plural of SNP. Of course, when one refers to DNA herein such reference may include derivatives of DNA such as amplicons, RNA transcripts, etc.

As used herein, the term “SNP haplotype block” means a group of variant or SNP locations that do not appear recombine independently and that can be grouped together in blocks of variants or SNPs.

As used herein, the term “SNP haplotype pattern” refers to the set of genotypes for SNPs in a SNP haplotype block in a single DNA strand.

As used herein, the term “SNP location” is the site in a DNA sequence where a SNP occurs.

As used herein a “SNP haplotype sequence” is a DNA sequence in a DNA strand that contains at least one SNP location.

Any reference made to DNA herein may include derivatives of DNA such as amplicons, RNA transcripts, nucleic acid mimetics, etc.

### Preparation of Nucleic Acids for Analysis

Nucleic acid molecules may be prepared for analysis using any technique known to those skilled in the art. Preferably such techniques result in the production of a nucleic acid molecule sufficiently pure to determine the presence or absence of one or more variations at one or more locations in the nucleic acid molecule. Such techniques may be found, for example, in Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), each of which is incorporated herein by reference.

When the nucleic acid of interest is present in a cell, it may be necessary to first prepare an extract of the cell and then perform further steps—i.e., differential precipitation, column chromatography, extraction with organic solvents and the like—in order to obtain a sufficiently pure preparation of nucleic acid. Extracts may be prepared using standard techniques in the art, for example, by chemical or mechanical lysis of the cell. Extracts then may be further treated, for example, by filtration and/or centrifugation and/or with chaotropic salts such as guanidinium isothiocyanate or urea or with organic solvents such as phenol and/or HCCl₃ to denature any contaminating and potentially interfering proteins. When chaotropic salts are used, it may be desirable to remove the salts from the nucleic acid-containing sample. This can be accomplished using standard techniques in the art such as precipitation, filtration, size exclusion chromatography and the like.

One approach particularly suitable for examining haplotype patterns and blocks is using somatic cell genetics to separate chromosomes from a diploid state to a haploid state. In one embodiment, a human lymphoblastoid cell line that is diploid may be fused to a hamster fibroblast cell line that is also diploid such that the human chromosomes are introduced into the hamster cells to produce cell hybrids. The resulting cell hybrids are examined to determine which human chromosomes were transferred, and which, if any, of the transferred human chromosomes are in a haploid state (see, e.g., Patterson, et al., Annal. N.Y. Acad. Of Sciences, 396:69-81 (1982)).

### Amplification Techniques

It may be desirable to amplify one or more nucleic acids of interest before determining the presence or absence of one or more variations in the nucleic acid. Nucleic acid amplification increases the number of copies of the nucleic acid sequence of interest. Any amplification technique known to those of skill in the art may be used in conjunction with the present invention including, but not limited to, polymerase chain reaction (PCR) techniques. PCR may be carried out using materials and methods known to those of skill in the art.

Techniques to optimize the amplification of long sequences may be used. Such techniques work well on genomic sequences. The methods disclosed in pending US patent applications U.S. Ser. No. 10/042,406, filed Jan. 9, 2002 entitled “Algorithms for Selection of Primer Pairs”; and U.S. Ser. No. 10/042,492, filed Jan. 9, 2002, entitled “Methods for Amplification of Nucleic Acids”, both assigned to the assignee of the present invention, are particularly suitable for amplifying genomic DNA for use in the methods of the present invention.

Amplified sequences may be subjected to other post amplification treatments either before or after labeling. For example, in some cases, it may be desirable to fragment the amplified sequence prior to hybridization with an oligonucleotide array. Fragmentation of the nucleic acids generally may be carried out by physical, chemical or enzymatic methods that are known in the art. Suitable techniques include, but are not limited to, subjecting the amplified nucleic acids to shear forces by forcing the nucleic acid containing fluid sample through a narrow aperture or digesting the PCR product with a nuclease enzyme. One example of a suitable nuclease enzyme is Dnase I. Labeling may be accomplished with any detectable group known in the art, for example, using fluorescent groups, ligands and/or radioactive groups.

### Methods for the Detection of SNPs

Determination of the presence or absence of one or more variations in a nucleic acid may be made using any technique known to those of skill in the art. Any technique that permits the accurate determination of a variation can be used. Preferred techniques will permit rapid, accurate determination of multiple variations with a minimum of sample handling. Some examples of suitable techniques are provided below.

Several methods for DNA sequencing are well known and generally available in the art and may be used to determine the location of SNPs in a genome. See, for example, Sambrook, et al., Molecular Cloning: A Laboratory Manual (Cold Spring Harbor Laboratory, New York) (1989), and Ausubel, et al., Current Protocols in Molecular Biology (John Wiley and Sons, New York) (1997), both of which are incorporated herein by reference. Such methods may be used to determine the sequence of the same genomic regions from different DNA strands where the sequences are then compared and the differences (variations between the strands) are noted. DNA sequencing methods may employ such enzymes as the Klenow fragment of DNA polymerase I, Sequenase (US Biochemical Corp, Cleveland, Ohio.), Taq polymerase (Perkin Elmer), thermostable T7 polymerase (Amersham, Chicago, Ill.), or combinations of polymerases and proofreading exonucleases such as those found in the Elongase Amplification System marketed by Gibco/BRL (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA Sequencers (Perkin Elmer, Wellesley, Mass.). In addition, capillary electrophoresis systems which are commercially available may be used to perform variation or SNP analysis.

Optionally, once a genomic sequence from one reference DNA strand has been determined by sequencing, it is possible to use hybridization techniques to determine variations in sequence between the reference strand and other DNA strands. These variations may be SNPs. An example of a suitable hybridization technique involves the use of DNA chips (oligonucleotide arrays), for example, those available from Affymetrix, Inc., Santa Clara, Calif. For details on the use of DNA chips for the detection of, for example, SNPs, see U.S. Pat. No. 6,300,063 issued to Lipshultz, et al., and U.S. Pat. No. 5,837,832 to Chee, et al., HuSNP Mapping Assay, reagent kit and user manual, Affymetrix Part No. 90094 (Affymetrix, Santa Clara, Calif.), all incorporated by reference.

In some embodiments, more than 10,000 bases of a reference sequence and the other DNA strands are scanned for variants, and up more than 1×10⁹ bases of a reference sequence and the other DNA strands may be scanned for variants. Generally, at least exons are scanned for variants, and preferably both introns and exons are scanned for variants. Even more preferably, introns, exons and intergenic sequences are scanned for variants. The scanned nucleic acids may be genomic DNA, including both coding and noncoding regions, from a mammalian organism such as a human. Generally, more than 50% of the genomic DNA from the organism is scanned, and preferably, more than 75% of the genomic DNA is scanned. In some embodiments of the present invention, known repetitive regions of the genome are not scanned, and do not count toward the percentage of genomic DNA scanned. Such known repetitive regions may include Single Interspersed Nuclear Elements (SINEs, such as alu and MIR sequences), Long Interspersed Nuclear Elements (LINEs, such as LINE1 and LINE2 sequences), Long Terminal Repeats (LTRs such as MaLRs, Retrov and MER4 sequences), transposons, and MER1 And MER2 sequences.

Briefly, in one embodiment, labeled nucleic acids in a suitable solution are denatured—for example, by heating to 95° C.—and the solution containing the denatured nucleic acids is incubated with a DNA chip. After incubation, the solution is removed, the chip may be washed with a suitable washing solution to remove unhybridized nucleic acids, and the presence of hybridized nucleic acids on the chip is detected. The stringency of the wash conditions may be adjusted as necessary to produce a stable signal. Detecting the hybridized nucleic acids may be done directly, for example, if the nucleic acids contain a fluorescent reporter group, fluorescence may be directly detected. If the label on the nucleic acids is not directly detectable, for example, biotin, then a solution containing a detectable label, for example, streptavidin coupled to phycoerythrin, may be added prior to detection. Other reagents designed to enhance the signal level may also be added prior to detection, for example, a biotinylated antibody specific for streptavidin may be used in conjunction with the biotin, streptavidin-phycoerythrin detection system.

Once variant locations have been determined (SNP discovery) by using, for example, sequencing or microarray analysis, it is necessary to genotype the SNPs of control and sample populations. The hybridization methods just described work well for this purpose, providing an accurate and rapid technique for detecting and genotyping SNPs in multiple samples. Alternatively, a technique suitable for the detection of SNPs in genomic DNA—without amplification—is the Invader technology available from Third Wave Technologies, Inc., Madison, Wis. Use of this technology to detect SNPs may be found, e.g., in Hessner, et al., Clinical Chemistry 46(8):1051-56 (2000); Hall, et al., PNAS 97(15):8272-77 (2000); Agarwal, et al., Diag. Molec. Path. 9(3):158-64 (2000); and Cooksey, et al., Antimicrobial and Chemotherapy 44(5):1296-1301 (2000). In the Invader process, two short DNA probes hybridize to a target nucleic acid to form a structure recognized by a nuclease enzyme. For SNP analysis, two separate reactions are run—one for each SNP variant. If one of the probes is complementary to the sequence, the nuclease will cleave it to release a short DNA fragment termed a “flap”. The flap binds to a fluorescently-labeled probe and forms another structure recognized by a nuclease enzyme. When the enzyme cleaves the labeled probe, the probe emits a detectable fluorescence signal thereby indicating which SNP variant is present.

An alternative to Invader technology, rolling circle amplification utilizes an oligonucleotide complementary to a circular DNA template to produce an amplified signal (see, for example, Lizardi, et al., Nature Genetics 19(3):225-32 (1998); and Zhong, et al., PNAS 98(7):3940-45 (2001)). Extension of the oligonucleotide results in the production of multiple copies of the circular template in a long concatemer. Typically, detectable labels are incorporated into the extended oligonucleotide during the extension reaction. The extension reaction can be allowed to proceed until a detectable amount of extension product is synthesized.

Another technique suitable for the detection of SNPs makes use of the 5′-exonuclease activity of a DNA polymerase to generate a signal by digesting a probe molecule to release a fluorescently labeled nucleotide. This assay is frequently referred to as a Taqman assay (see, e.g., Arnold, et al., BioTechniques 25(1):98-106 (1998); and Becker, et al., Hum. Gene Ther. 10:2559-66 (1999)). A target DNA containing a SNP is amplified in the presence of a probe molecule that hybridizes to the SNP site. The probe molecule contains both a fluorescent reporter-labeled nucleotide at the 5′-end and a quencher-labeled nucleotide at the 3′-end. The probe sequence is selected so that the nucleotide in the probe that aligns with the SNP site in the target DNA is as near as possible to the center of the probe to maximize the difference in melting temperature between the correct match probe and the mismatch probe. As the PCR reaction is conducted, the correct match probe hybridizes to the SNP site in the target DNA and is digested by the Taq polymerase used in the PCR assay. This digestion results in physically separating the fluorescent labeled nucleotide from the quencher with a concomitant increase in fluorescence. The mismatch probe does not remain hybridized during the elongation portion of the PCR reaction and is, therefore, not digested and the fluorescently labeled nucleotide remains quenched.

Denaturing HPLC using a polystyrene-divinylbenzene reverse phase column and an ion-pairing mobile phase can be used to identify SNPs. A DNA segment containing a SNP is PCR amplified. After amplification, the PCR product is denatured by heating and mixed with a second denatured PCR product with a known nucleotide at the SNP position. The PCR products are annealed and are analyzed by HPLC at elevated temperature. The temperature is chosen to denature duplex molecules that are mismatched at the SNP location but not to denature those that are perfect matches. Under these conditions, heteroduplex molecules typically elute before homoduplex molecules. For an example of the use of this technique see Kota, et al., Genome 44(4):523-28 (2001).

SNPs also can be detected using solid phase amplification and microsequencing of the amplification product. Beads to which primers have been covalently attached are used to carry out amplification reactions. The primers are designed to include a recognition site for a Type II restriction enzyme. After amplification—which results in a PCR product attached to the bead—the product is digested with the restriction enzyme. Cleavage of the product with the restriction enzyme results in the production of a single stranded portion including the SNP site and a 3′-OH that can be extended to fill in the single stranded portion. Inclusion of ddNTPs in an extension reaction allows direct sequencing of the product. For an example of the use of this technique to identify SNPs see Shapero, et al., Genome Research 11(11):1926-34 (2001).

### Analysis to Establish SNP Haplotype Blocks and Patterns and Informative SNPs

The present invention is drawn to analysis of SNPs for association studies. All SNPs may by used; alternatively, a subset of SNPs, for example, common SNPs or informative SNPs selected from a set of common SNPs might be assayed. The methods and materials for variant discovery and data analysis described herein are also described in detail in priority documents U.S. provisional patent application serial No. 60/280,530, filed Mar. 30, 2001, U.S. provisional patent application serial No. 60/313,264 filed Aug. 17, 2001, U.S. provisional patent application serial No. 60/327,006, filed Oct. 5, 2001, all entitled “Identifying Human SNP Haplotypes, Informative SNPs and Uses Thereof”, provisional patent application serial No. 60/337/567 filed Nov. 30, 2001 and U.S. utility patent application Ser. No. 10/106,097, filed Mar. 26, 2002, both entitled “Methods for Genomic Analysis”.

### Business Methods for Development of Therapeutics and Diagnostic Products

The present invention provides business methods for the development of therapeutic and diagnostic products with the use of genetic information. Specifically, the invention provides using SNP haplotype pattern information derived from essentially coding regions of individuals in a control group and individuals in a case group and comparing the frequencies of the SNP haplotype patterns of the two groups to identifiy genetic loci related to a phenotype of interest where the phenotype is present in the case group and not in the control group. The methods further provide using the identified genetic loci in a discovery process for the discovery and development of therapeutic and diagnostic products. Business methods using genetic information are described in detail in co-owned pending application U.S. Ser. No. 10/107,508 filed Mar. 26, 2003, the disclosure of which is incorporated by reference in its entirety for all purposes herein.

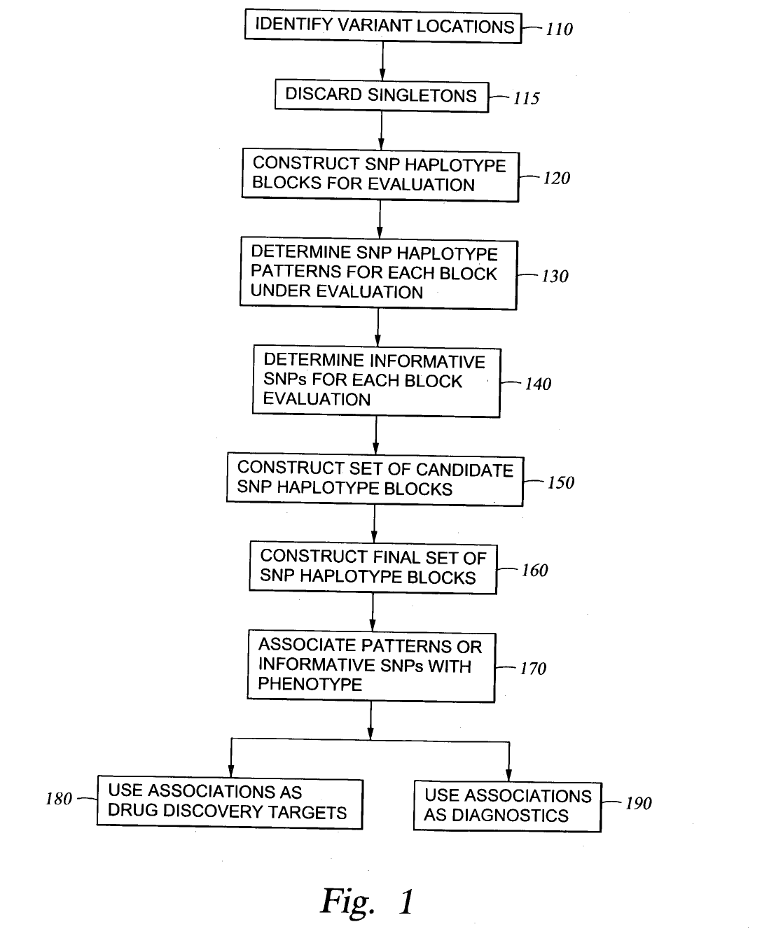

FIG. 1 is a schematic showing the steps of one embodiment of the methods of the present invention. Once SNPs (variants) have been located or discovered (step 110 of FIG. 1) by, e.g., the methods described supra, SNP haplotype blocks, SNP haplotype patterns within each SNP haplotype block, and informative SNPs for the SNP haplotype patterns may be determined. One may use all SNPs or variants located; alternatively, one may focus the analysis on only a portion of the SNPs located. For example, the set of SNPs analyzed may exclude singleton SNPs. Singleton SNPs are specific SNP alleles that occur in less than a certain portion of the samples on which SNP discovery is performed. Thus, singleton SNPs are those that occur in, for example, less than 20%, 10%, 5% or 1% of samples assayed, depending on the parameters selected. A related concept is that of common SNPs. Common SNPs are those SNPs where the less common form is present at a minimum frequency in a given population. For example, common SNPs are those SNPs that are found in at least about 2% to 25% of the population, and, generally, common SNPs are those SNPs that are found in at least about 10% of the population. Common SNPs likely result from mutations that occurred early in the evolution of humans, where singleton SNPs are those SNPs that likely result from mutations that occurred relatively recently in evolution. Step 115 of FIG. 1 shows that, in the present invention, singleton SNPs are discarded from SNP haplotype block and pattern analysis.

In step 120 of FIG. 1, the common variants or SNPs of interest are assigned to haplotype blocks for evaluation. Common variants or SNPs from a whole genome or chromosome may be analyzed and assigned to SNP haplotype blocks. Alternatively, common variants from only a focused genomic region specific to some disease or drug response mechanism may be assigned to the SNP haplotype blocks.

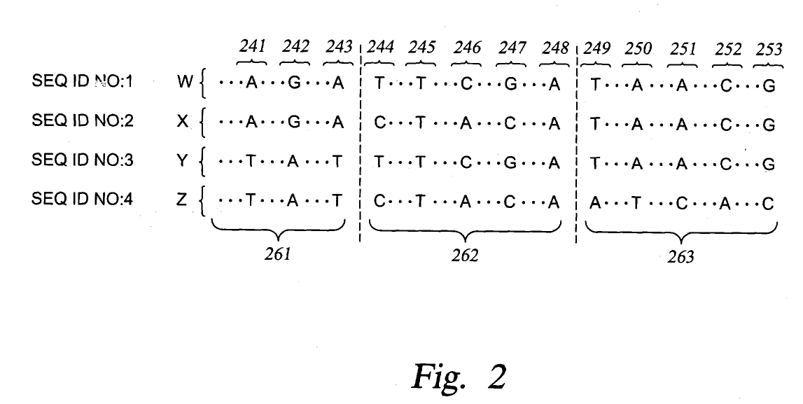

FIG. 2 provides one illustration of showing how common variants, usually SNPs, occur in haplotype blocks in a genome, and that more than one haplotype pattern can occur within each haplotype block. If SNP haplotype patterns were completely random, it would be expected that the number of possible SNP haplotype patterns observed for a SNP haplotype block of N SNPs would be 2^{N} (not 4^{N}, as the variants will most commonly be biallelic, i.e., occur in only one of two forms, not all four nucleotide base possibilities). However, it was observed in performing the methods of the present invention that the number of SNP haplotype patterns in each SNP haplotype block is smaller than 2^{N} because the SNPs are linked. Certain SNP haplotype patterns were observed at a much higher frequency than would be expected in a non-linkage case. Thus, SNP haplotype blocks are chromosomal regions that tend to be inherited as a unit, with a relatively small number of common patterns. Each line in FIG. 2 represents portions of the haploid genome sequence of different individuals. As shown therein, individual W has an “A” at position 241, a “G” at position 242, and an “A” at position 243. Individual X has the same bases at positions 241, 242, and 243. Conversely, individual Y has a T at positions 241 and 243, but an A at position 242. Individual Z has the same bases as individual Y at positions 241, 242, and 243. Variants in block 261 will tend to occur together. Similarly, the variants in block 262 will tend to occur together, as will those variants in block 263. Of course, only a few bases in a genome are shown in FIG. 2. In fact, most bases will be like those at position 245 and 248, and will not vary from individual to individual.

The assignment of common or non-singleton SNPs to SNP haplotype blocks, step 120 of FIG. 1, is, in one case, an iterative process involving the construction of SNP haplotype blocks from the SNP locations along a genomic region of interest. In one embodiment, once the initial SNP haplotype blocks are constructed, SNP haplotype patterns present in the constructed SNP haplotype blocks are determined (step 130 of FIG. 1). In some specific embodiments, the number of SNP haplotype patterns selected per SNP haplotype block in step 130 is no greater than about five. In another specific embodiment, the number of SNP haplotype patterns selected per SNP haplotype block is equal to the number of SNP haplotype patterns necessary to identify SNP haplotype patterns in greater than 80% of the DNA strands being analyzed. In some embodiments of the present invention, SNP haplotype patterns that occur in less than a certain portion of DNA strands being analyzed (singleton SNP haplotypes) are eliminated from analysis. For example, in one embodiment, if twenty DNA strands are being analyzed, SNP haplotype patterns that are found to occur in only one sample out of twenty are eliminated from analysis.

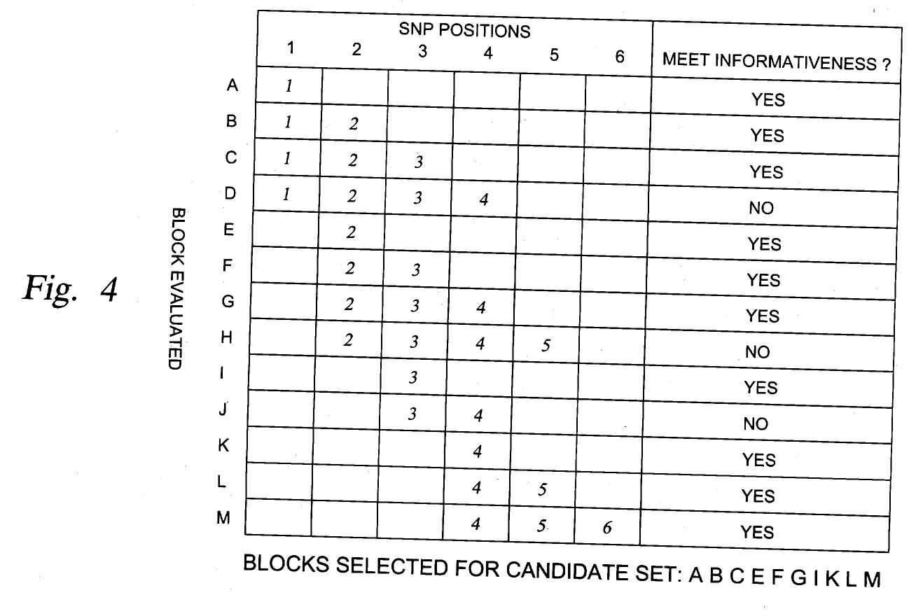

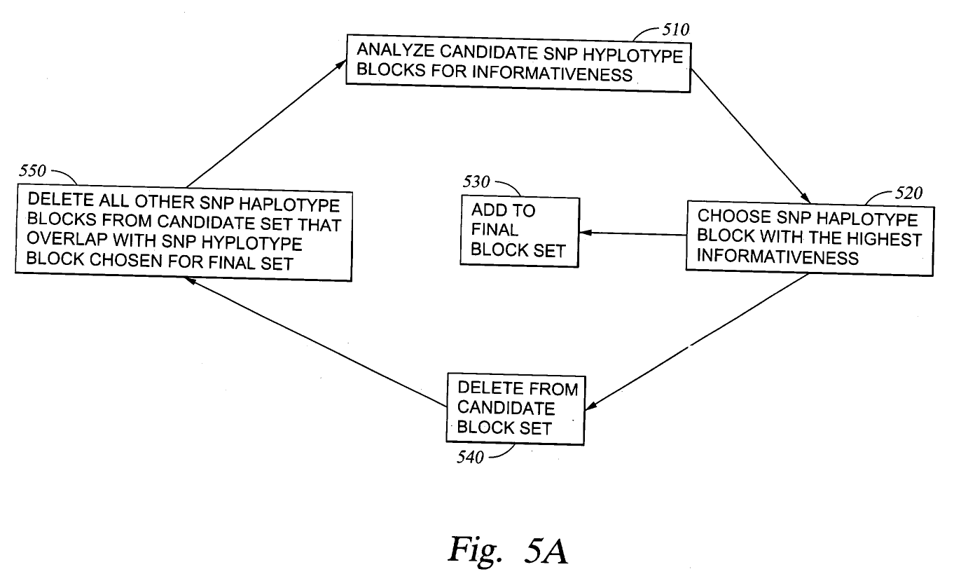

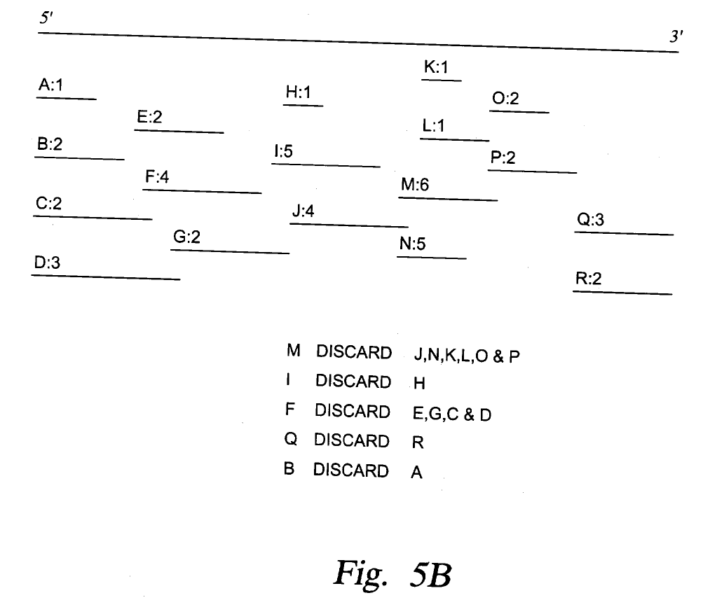

Once the SNP haplotype patterns of interest are selected, informative SNPs for these SNP haplotype patterns are determined (step 140 of FIG. 1). From this initial set of blocks, a set of candidate SNP blocks that fit certain criteria for informativeness is constructed (step 150 of FIG. 1). FIGS. 4 and 5 illustrate steps 120, 130, 140 and 150 in more detail.

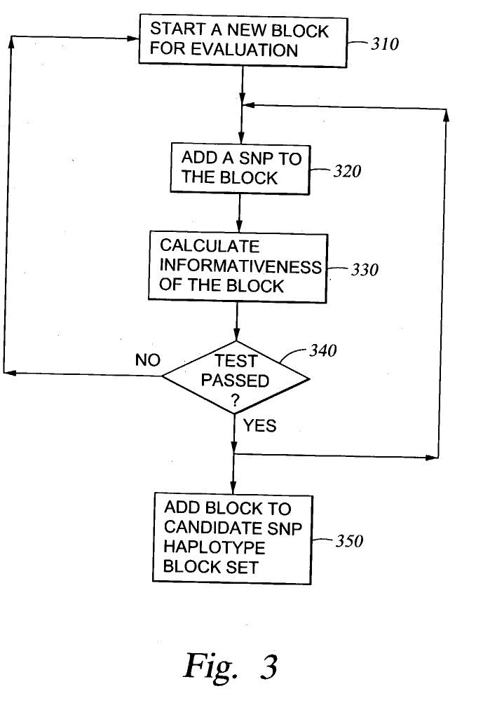

In FIG. 3, step 310 provides that a new block of SNPs is chosen for evaluation. In one embodiment, the first block chosen contains only the first SNP in a SNP haplotype sequence; thus at step 320, the first, single SNP is added to the block. At step 330, informativeness of this block is determined.

“Informativeness” of a SNP haplotype block is defined in one embodiment as the degree to which the block provides information about genetic regions. For example, in one embodiment of the present invention, informativeness could be calculated as the ratio of the number of SNP locations in a SNP haplotype block divided by the number of SNPs required to distinguish each SNP haplotype pattern under consideration from other SNP haplotype patterns under consideration (number of informative SNPs) in that block. Another measure of informativeness might be the number of informative SNPs in the block. One skilled in the art recognizes that informativeness may be determined in any number of ways.

Referring again to FIG. 2, SNP haplotype block 261 contains three SNPs and two SNP haplotype patterns (AGA and TAT). Any one of the three SNPs present can be used to tell the patterns apart; i.e., any one of these SNPs can be chosen to be the informative SNP for this SNP haplotype pattern. For example, if it is determined that a sample nucleic acid contains a T at the first position, the same sample will contain an A at the second position and a T at the third position. If it is determined in a second sample that the SNP in the second position is a G, the first and third SNPs will be A's. Thus, by one measure of informativeness, the informativeness value for this first block is 3: 3 total SNPs divided by 1 informative SNP needed to distinguish the patterns from each other. Similarly, SNP haplotype block 262 contains three SNPs (two positions do not have variants) and two haplotype patterns (TCG and CAC). As with the previously-analyzed block, any one of the three SNPs can be evaluated to tell one pattern from the other; thus, the informativeness of this block is 3: 3 total SNPs divided by 1 informative SNP needed to distinguish the patterns. SNP haplotype block 263 contains five SNPs and two SNP patterns (TAACG and ATCAC). Again, any one of the five SNPs can be used to tell one pattern from the other; thus, the informativeness of this block is 5: 5 total SNPs divided by 1 informative SNP needed to distinguish the patterns.

FIG. 2 provides a simple example of genetic analysis. When several SNP haplotype patterns are present in a block, it may be necessary to use more than one SNP as informative SNPs. For example, in a case where a block contains, for example, six SNPs and two SNPs are needed to distinguish the patterns of interest, the informativeness of the block is 3: 6 total SNPs divided by 2 SNPs needed to distinguish the patterns. Generally speaking, as many as 2^{N} distinct SNP haplotype patterns can be distinguished by using the genotypes of N suitably selected SNPs. Therefore, if there exist only two SNP haplotype patterns in the SNP haplotype block, a single SNP should be able to differentiate between the two. If there are three or four patterns, at least two SNPs would likely be required, etc.

In step 340 of FIG. 3, once the informativeness of a SNP haplotype block is determined, a test is performed. The test essentially evaluates the SNP haplotype blocks based on selected criteria (for example, whether a block meets a threshold measure of informativeness), and the result of the test determines whether, for example, another SNP will be added to the block for analysis or whether the analysis will proceed with a new block starting at a different SNP location. FIG. 4 illustrates one embodiment of this process.

In FIG. 4, assume there is a DNA sequence with six SNP locations. The analysis of SNP haplotype blocks described above might be performed in the following manner: SNP haplotype block A is selected containing only the SNP at SNP position 1 (steps 310 and 320 of FIG. 3). The informativeness of this block is calculated (step 330), and it is determined whether the informativeness of this block meets a threshold measure of informativeness (step 340). In this case, it “passes” and two things happen. First, this block of one SNP (SNP position 1) is added to the set of candidate SNP haplotype blocks (step 350). Second, another SNP (here, SNP position 2) is added to this block (step 320) to create a new block, B, containing SNP positions 1 and 2, which is then analyzed. In this illustration block B also meets the threshold measure of informativeness (step 340), so it would be added to the set of candidate SNP haplotype blocks (step 350), and another SNP (here, SNP position 3) is added to this block (step 320) to create new block C, containing SNP positions 1, 2 and 3, which is then analyzed. In this illustration, C also meets the threshold measure of informativeness and it is added to the set of candidate SNP haplotype blocks (step 350), and another SNP (here, SNP position 4) is added to this block (step 320) to create new block D, containing SNP positions 1, 2, 3, and 4, which is then analyzed. In the FIG. 4 illustration, SNP block D does not meet the threshold measure of informativeness. SNP block D is not added to the set of candidate SNP haplotype blocks (step 350), nor does another SNP get added to block D for analysis. Instead, a new SNP location is selected for a round of SNP block evaluations.

In FIG. 4, after block D fails to meet the threshold measure of informativeness, a new block, E, is selected that contains only the SNP at position 2. Block E is evaluated for informativeness, is found to meet the threshold measure, is added to the set of candidate SNP haplotype blocks (step 350), and another SNP (here, SNP position 3) is added to this block (step 320) to create new block F, containing SNP positions 2 and 3, which is then analyzed, and so on. Note that block H fails to meet the threshold measure of informativeness, is not added to the set of candidate SNP haplotype blocks (step 350), nor does another SNP get added to block H for analysis. Instead, a new block, 1, is selected that contains only the SNP at position 3, and so on.

Once a set of candidate SNP blocks is constructed (step 350 of FIG. 3), analysis is performed on the set to select a final set of SNP blocks (step 160 of FIG. 1). The selection of the final set of SNP blocks can performed in a variety of ways. For example, referring back to FIG. 4, one could select the largest block containing SNP position 1 that passes the threshold test (block C, containing SNPs 1, 2 and 3), discard the smaller blocks that contain the same SNPs (blocks A and B). Then the next block selected might be the next block starting with SNP position 4 that is the largest block that meets the threshold test for informativeness (block G) and the smaller blocks that contain the same SNPs (blocks E and F) would be discarded. Such a method would give a set of non-overlapping SNP haplotype blocks that span the genomic region of interest, contain the SNPs of interest and that have a high level of informativeness. Thus, once all candidate SNP haplotype blocks are evaluated, the result may be a set of non-overlapping SNP haplotype blocks that encompasses all the SNPs in the original set. Some blocks, called isolates, may consist of only a single SNP, and by definition have an informativeness of 1. Other groups may consist of a hundred or more SNPs, and have an informativeness exceeding 30.

An alternative method for selecting a final set of SNP haplotype blocks is shown in FIGS. 5A and 5B. This alternative method implements a greedy algorithm. Looking first at FIG. 5A, in a first step 510, the candidate SNP haplotype block set (generated, for example, by the methods described in FIGS. 3 and 4 herein) is analyzed for informativeness. In step 520, the candidate SNP haplotype block with the highest informativeness in the entire candidate set is chosen to be added to the final SNP haplotype block set (step 530). Once this candidate SNP haplotype block is chosen to be a member of the final SNP haplotype block set, it is deleted from the candidate block set (step 540), and all other candidate SNP haplotype blocks that overlap with the chosen block are deleted from the candidate SNP haplotype block set (step 550). Next, the candidate SNP haplotype blocks remaining in the candidate set are analyzed for informativeness (step 510), and the candidate SNP haplotype block with the highest informativeness is chosen to be added to the final SNP haplotype block set (steps 520 and 530). As before, once this SNP haplotype block is chosen to be a member of the final SNP haplotype block set, it is deleted from the candidate block set (step 540), and all other candidate SNP haplotype blocks that overlap with the chosen block are deleted from the candidate SNP haplotype block set (step 550). The process continues until a final set of non-overlapping SNP haplotype blocks that encompasses all the SNPs in the original set is constructed.

FIG. 5B illustrates a simple employment of the method of selecting a final set of SNP haplotype blocks described in FIG. 5A. In FIG. 5B, a sequence 5′ to 3′ is analyzed for SNPs, SNP haplotype patterns and candidate SNP haplotype blocks according to the methods of the present invention. Candidate SNP haplotype blocks contained within this sequence are indicated by their placement under the sequence, and are designated by a letter. In addition, after the letter, the informativeness of each block is indicated. For example, candidate SNP haplotype block A is located at the extreme 5′ end of the sequence, and has an informativeness of 1. Candidate SNP haplotype block R is located at the extreme 3′ end of the sequence, and has an informativeness of 2.

According to FIG. 5A, in a first step 510, the candidate SNP haplotype blocks are analyzed for informativeness, and in step 520, the SNP haplotype block with the highest informativeness is chosen to be added to the final SNP haplotype block set (steps 520 and 530). In the case of FIG. 5B, candidate SNP haplotype block M with an informativeness of 6 would be the first candidate SNP haplotype block selected to be added to the final SNP haplotype block set. Once SNP haplotype block M is selected, it is deleted or removed from the candidate set of SNP haplotype blocks (step 540), and all other candidate SNP haplotype blocks that overlap with SNP haplotype block M (blocks J, N, K, L, O and P) are deleted from the candidate SNP haplotype block set (step 550). Next, the remaining blocks of the candidate SNP haplotype block set, namely SNP haplotype blocks A, B, C, D, E, F, G, H, I, Q and R are analyzed for informativeness, and in step 520, the remaining SNP haplotype block with the highest informativeness, 1, with an informativeness of 5, is chosen to be added to the final SNP haplotype block set (530) and deleted or removed from the candidate set of SNP haplotype blocks (step 540). Next, in step 550, all other candidate SNP haplotype blocks that overlap with SNP haplotype block I, here, only block H, is deleted from the candidate SNP haplotype block set. Again, the remaining blocks of the candidate SNP haplotype block set, namely SNP haplotype blocks A, B, C, D, E, F, G, Q and R are analyzed for informativeness. In step 520, the remaining SNP haplotype block with the highest informativeness, block F, with an informativeness of 4, is chosen to be added to the final SNP haplotype block set (530) and deleted or removed from the candidate set of SNP haplotype blocks (step 540). Next, all other candidate SNP haplotype blocks that overlap with SNP haplotype block F—here, blocks E, G, C and D—are deleted from the candidate SNP haplotype block set, and the remaining blocks of the candidate SNP haplotype block set, namely SNP haplotype blocks A, B, Q and R, are analyzed for informativeness, and so on.

Other methods may be employed to select a final set of SNP haplotype blocks for analysis from the set of candidate SNP haplotype blocks (step 160 of FIG. 1). Algorithms known in the art may be applied for this purpose. For example, shortest-paths algorithms or other dynamic algorithms may be used (see, generally, Cormen, Leiserson, and Rivest, Introduction to Algorithms (MIT Press) pp. 514-78 (1994)). Such algorithms can be used to evaluate the variants to produce a set of, preferably, non-overlapping SNP haplotype blocks that encompasses all SNPs evaluated in a particular genomic region. An important result of selecting SNPs, SNP haplotype blocks and SNP haplotype patterns according to the methods of the present invention is that in some embodiments during the calculation of informativeness of SNP haplotype blocks, informative SNPs for each SNP haplotype block and pattern are determined. Informative SNPs allow for data compression.

### Association of Phenotypes with SNP Haplotypes Blocks and Patterns

The SNP haplotype blocks, SNP haplotype patterns and/or informative SNPs identified may be used in a variety of genetic analyses. For example, once informative SNPs have been identified in the SNP haplotype patterns, they may be used in a number of different assays for association studies. For example, probes may be designed for microarrays that interrogate these informative SNPs. Other exemplary assays include, e.g., the Taqman assays and Invader assays described supra, as well as conventional PCR and/or sequencing techniques.

In some embodiments, as shown in step 170 of FIG. 1, the haplotype patterns identified may be used in the above-referenced assays to perform association studies. This may be accomplished by determining haplotype patterns in individuals with the phenotype of interest (for example, individuals exhibiting a particular disease or individuals who respond in a particular manner to administration of a drug) and comparing the frequency of the haplotype patterns in these individuals to the haplotype pattern frequency in a control group of individuals. Preferably, such SNP haplotype pattern determinations are genome-wide; however, it may be that only specific regions of the genome are of interest, and the SNP haplotype patterns of those specific regions are used. In addition to the other embodiments of the methods of the present invention disclosed herein, the methods additionally allow for the “dissection” of a phenotype. That is, a particular phenotype may result from two or more different genetic bases. For example, obesity in one individual may be the result of a defect in Gene X, while the obesity phenotype in a different individual may be the result of mutations in Gene Y and Gene Z. Thus, the genome scanning capabilities of the present invention allow for the dissection of varying genetic bases for similar phenotypes. Once specific regions of the genome are identified as being associated with a particular phenotype, these regions may be used as drug discovery targets (step 180 of FIG. 1) or as diagnostic markers or used for further analysis (step 190 of FIG. 1).

As described in the previous paragraph, one method of conducting association studies is to compare the frequency of SNP haplotype patterns in individuals with a phenotype of interest to the SNP haplotype pattern frequency in a control group of individuals. In a preferred method, informative SNPs are used to make the SNP haplotype pattern comparison. The approach of using informative SNPs has tremendous advantage over other whole genome scanning or genotyping methods known in the art to date, for instead of reading all 3 billion bases of each individual's genome—or even reading the 3-4 million common SNPs that may be found—only informative SNPs from a sample population need to be determined. Reading these particular, informative SNPs provides sufficient information to allow statistically accurate association data to be extracted from specific experimental populations, as described above.

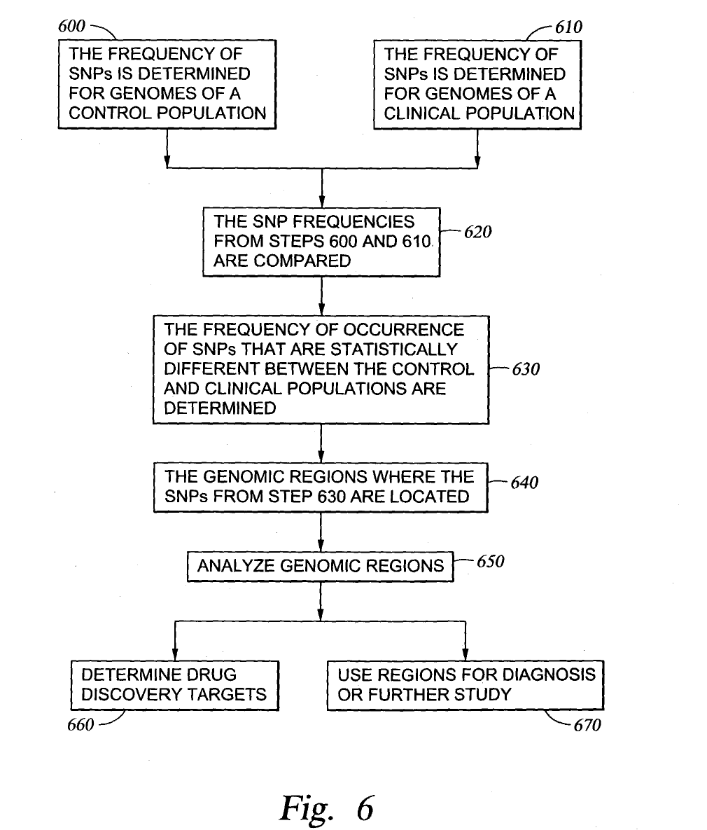

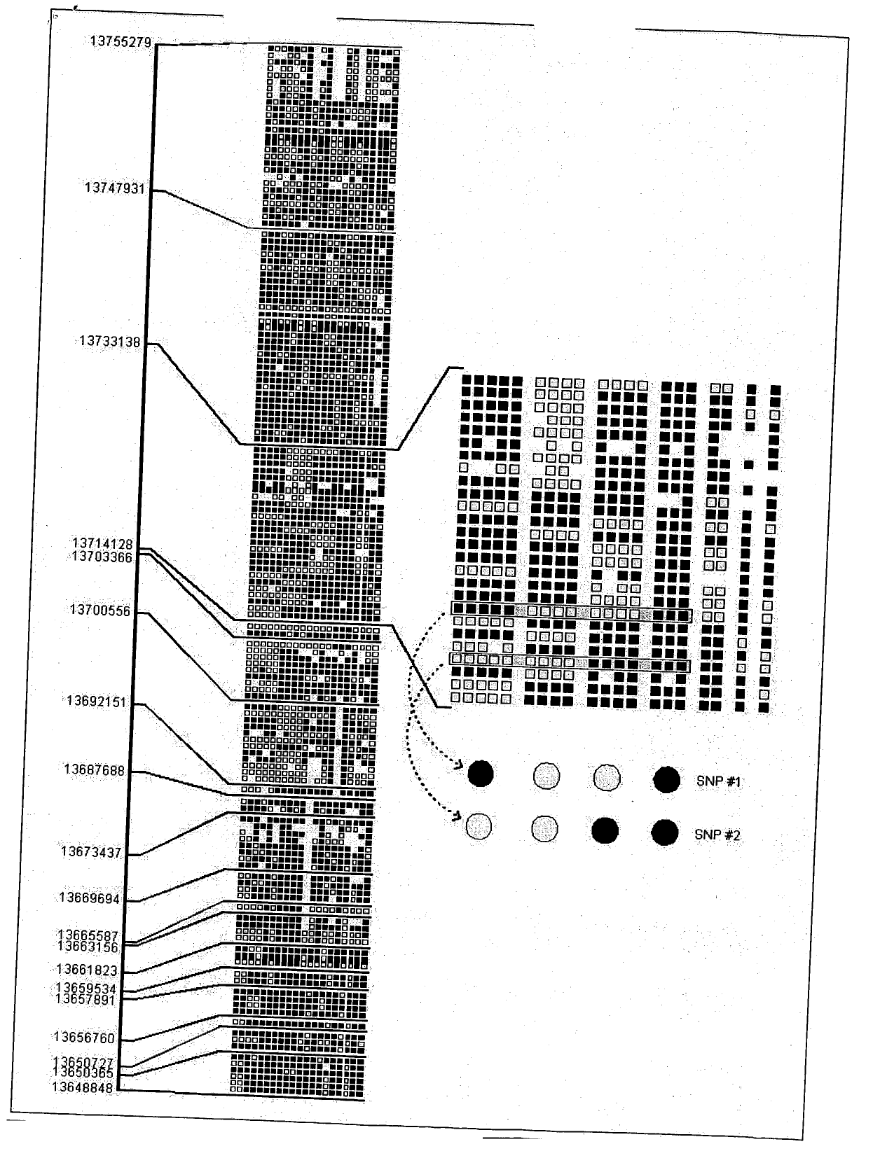

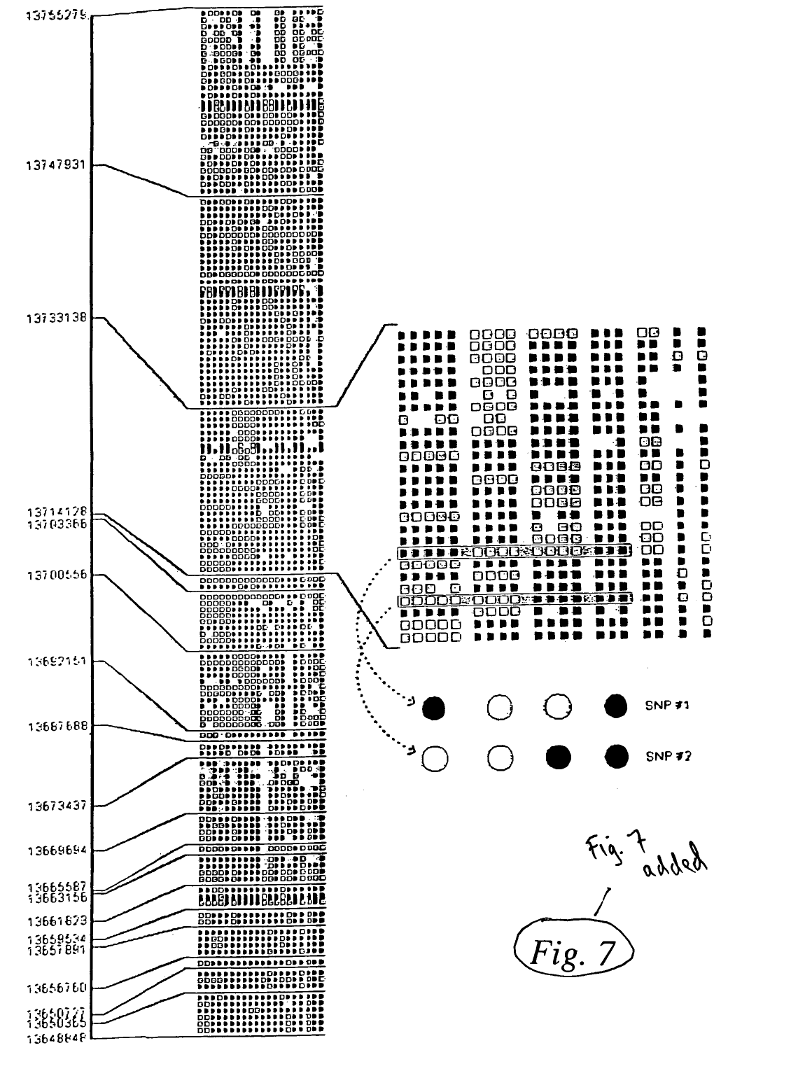

FIG. 6 illustrates an embodiment of one method of determining genetic associations using the methods of the present invention. In step 600, the frequency of informative SNPs is determined for genomes of a control population. In step 610, the frequency of informative SNPs is determined for genomes of a clinical population. Steps 600 and 610 may be performed by using the aforementioned SNP assays to analyze the informative SNPs in a population of individuals. In step 620, the informative SNP frequencies from steps 600 and 610 are compared. Frequency comparisons may be made, for example, by determining the minor allele frequency (number of individuals with a particular minor allele divided by the total number of individuals) at each informative SNP location in each population and comparing these minor allele frequencies. In step 630, the informative SNPs displaying a difference between the frequency of occurrence in the control versus clinical populations are selected for analysis. Once informative SNPs are selected, the SNP haplotype blocks that contain the informative SNPs are identified, which in turn identifies the genomic region of interest (step 640). The genomic regions are analyzed by genetic or biological methods known in the art (step 650), and the regions are analyzed for possible use as drug discovery targets (step 660) or as diagnostic markers (step 670), as described in detail below.

### Uses of Identified Genomic Sequences

Once a genetic locus or multiple loci in the genome are associated with a particular phenotypic trait—for example, a disease susceptibility locus—the gene or genes or regulatory elements responsible for the trait can be identified. These genes or regulatory elements may then be used as therapeutic targets for the treatment of the disease, as shown in step 180 of FIG. 1 or step 660 of FIG. 6. To determine how the genes identified may be used to treat disease, the sequence of the gene, including flanking promoter regions and coding regions, may be mutated in various ways known in the art to generate targeted changes in expression level, or changes in the sequence of the encoded protein, etc. The sequence changes may be substitutions, insertions, translocations or deletions. Deletions may include large changes, such as deletions of an entire domain or exon. Techniques for in vitro mutagenesis of cloned genes are known. Examples of protocols for site specific mutagenesis may be found in, e.g., Gustin, et al., Biotechniques 14:22 (1993) and Sambrook, et al., Molecular Cloning: A Laboratory Manual (Cold Spring Harbor Press) pp. 15.3-15.108 (1989. Such mutated genes may be used to study structure/function relationships of the protein product, or to alter the properties of the protein that affect its function or regulation.

The identified gene may be employed for producing all or portions of the resulting polypeptide. To express a protein product, an expression cassette incorporating the identified gene may be employed. The expression cassette or vector generally provides a transcriptional and translational initiation region, which may be inducible or constitutive, where the coding region is operably linked under the transcriptional control of the transcriptional initiation region, and a transcriptional and translational termination region. These control regions may be native to the identified gene, or may be derived from exogenous sources.

An expressed protein may be used for the production of antibodies, where short fragments induce the expression of antibodies specific for the particular polypeptide (monoclonal antibodies), and larger fragments or the entire protein allow for the production of antibodies over the length of the polypeptide (poyclonal antibodies). Antibodies are prepared in accordance with conventional ways, where the expressed polypeptide or protein is used as an immunogen, by itself or conjugated to known immunogenic carriers, e.g. KLH, pre-S HBsAg, other viral or eukaryotic proteins, or the like. For monoclonal antibodies, after one or more booster injections, the spleen is isolated, the lymphocytes are immortalized by cell fusion and screened for high affinity antibody binding. The immortalized cells, i.e, hybridomas, producing the desired antibodies may then be expanded. For further description, see Monoclonal Antibodies: A Laboratory Manual, Harlow and Lane, eds. (Cold Spring Harbor Laboratories, Cold Spring Harbor, N.Y.) (1988).

The identified genes, gene fragments, or the encoded protein or protein fragments may be useful in gene therapy to treat degenerative and other disorders. For example, expression vectors may be used to introduce the identified gene into a cell. Such vectors generally have convenient restriction sites located near the promoter sequence to provide for the insertion of nucleic acid sequences in a recipient genome. Transcription cassettes may be prepared comprising a transcription initiation region, the target gene or fragment thereof, and a transcriptional termination region. The transcription cassettes may be introduced into a variety of vectors, e.g. plasmid; retrovirus, e.g. lentivirus; adenovirus; and the like, where the vectors are able to be transiently or stably maintained in the cells. The gene or protein product may be introduced directly into tissues or host cells by any number of routes, including viral infection, microinjection, or fusion of vesicles.

Antisense molecules can be used to down-regulate expression of the identified gene in cells. The antisense reagent may be antisense oligonucleotides, particularly synthetic antisense oligonucleotides having chemical modifications, or nucleic acid constructs that express such antisense molecules as RNA. A combination of antisense molecules may be administered, where a combination may comprise multiple different sequences. As an alternative to antisense inhibitors, catalytic nucleic acid compounds, e.g., ribozymes, anti-sense conjugates, etc., may be used to inhibit gene expression.

Investigation of genetic function may also utilize non-mammalian models, particularly using those organisms that are biologically and genetically well-characterized, such as C. elegans, D. melanogaster and S. cerevisiae. The subject gene sequences may be used to knock-out corresponding gene function or to complement defined genetic lesions in order to determine the physiological and biochemical pathways involved in protein function. Drug screening may be performed in combination with complementation or knock-out studies, e.g., to study progression of degenerative disease, to test therapies, or for drug discovery.

Protein molecules may be assayed to investigate structure/function parameters. For example, by providing for the production of large amounts of a protein product of an identified gene, one can identify ligands or substrates that bind to, modulate or mimic the action of that protein product. Drug screening identifies agents that provide, e.g., a replacement or enhancement for protein function in affected cells, or for agents that modulate or negate protein function. The term “agent” as used herein describes any molecule, e.g. protein or small molecule, with the capability of altering, mimicking or masking, either directly or indirectly, the physiological function of an identified gene or gene product.

Candidate agents encompass numerous chemical classes, though typically they are organic molecules or complexes, preferably small organic compounds, having a molecular weight of more than 50 and less than about 2,500 daltons, and may be obtained from a wide variety of sources including libraries of synthetic or natural compounds.

Where the screening assay is a binding assay, one or more of the molecules may be coupled to a label, where the label can directly or indirectly provide a detectable signal. Various labels include radioisotopes, fluorescers, chemiluminescers, enzymes, specific binding molecules, particles, e.g., magnetic particles, and the like. Specific binding molecules include pairs, such as biotin and streptavidin, digoxin and antidigoxin, etc. For the specific binding members, the complementary member would normally be labeled with a molecule that provides for detection, in accordance with known procedures.

The SNPs identified by the present invention may be used to analyze the expression pattern of an associated gene and the expression pattern correlated to a phenotypic trait of the organism such as disease susceptibility or drug responsiveness. The expression pattern in various tissues can be determined and used to identify ubiquitous expression patterns, tissue specific expression patterns, temporal expression patterns and expression patterns induced by various external stimuli such as chemicals or electromagnetic radiation. Such determinations would provide information regarding function of the gene and/or its protein product. The newly identified sequences also may be used as diagnostic markers, i.e., to predict a phenotypic characteristic such as disease susceptibility or drug responsiveness. Moreover, when a phenotype cannot clearly distinguish between similar diseases having different genetic bases, the methods of the present invention can be used to identify correctly the disease.

Also, it should be apparent that the methods of the present invention can be used on organisms aside from humans. For example, when the organism is an animal, the methods of the invention may be used to identify loci associated, e.g., with disease resistance/or susceptibility, environmental tolerance, drug response or the like, and when the organism is a plant, the method of the invention may be used to identify loci associated with disease resistance/or susceptibility, environmental tolerance and or herbicide resistance.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

### EXAMPLE 1

### Preparation of Somatic Cell Hybrids

Standard procedures in somatic cell genetics were used to separate human DNA strands (chromosomes) from a diploid state to a haploid state. In this case, a diploid human lymphoblastoid cell line that was wildtype for the thymidine kinase gene was fused to a diploid hamster fibroblast cell line containing a mutation in the thymidine kinase gene. A sub-population of the resulting cells were hybrid cells containing human chromosomes. Hamster cell line A23 cells were pipetted into a centrifuge tube containing 10 ml DMEM in which 10% fetal bovine serum (FBS)+1×Pen/Strep+10% glutamine were added, centrifuged at 1500 rpm for 5 minutes, resuspended in 5 ml of RPMI and pipetted into a tissue culture flask containing 15 ml RPMI medium. The lymphoblastoid cells were grown at 37° C. to confluence. At the same time, human lymphoblastoid cells were pipetted into a centrifuge tube containing 10 ml RPMI in which 15% FBCS+1×Pen/Strep+10% glutamine were added, centrifuged at 1500 rpm for 5 minutes, resuspended in 5 ml of RPMI and pipetted into a tissue culture flask containing 15 ml RPMI. The lymphoblastoid cells were grown at 37° C. to confluence.

To prepare the A23 hamster cells, the growth medium was aspirated and the cells were rinsed with 10 ml PBS. The cells were then trypsinized with 2 ml of trypsin, divided onto 3-5 plates of fresh medium (DMEM without HAT) and incubated at 37° C. The lymphoblastoid cells were prepared by transferring the culture into a centrifuge tube and centrifuging at 1500 rpm for 5 minutes, aspirating the growth medium, resuspending the cells in 5 ml RPMI and pipetting 1 to 3 ml of cells into 2 flasks containing 20 ml RPMI.

To achieve cell fusion, approximately 8-10×10⁶ lymphoblastoid cells were centrifuged at 1500 rpm for 5 min. The cell pellet was then rinsed with DMEM by resuspending the cells, centrifuging them again and aspirating the DMEM. The lymphoblastoid cells were then resuspended in 5 ml fresh DMEM. The recipient A23 hamster cells had been grown to confluence and split 3-4 days before the fusion and were, at this point, 50-80% confluent. The old media was removed and the cells were rinsed three times with DMEM, trypsinized, and finally suspended in 5 ml DMEM. The lymphoblastoid cells were slowly pipetted over the recipient A23 cells and the combined culture was swirled slowly before incubating at 37° C. for 1 hour. After incubation, the media was gently aspirated from the A23 cells, and 2 ml room temperature PEG 1500 was added by touching the edge of the plate with a pipette and slowly adding PEG to the plate while rotating the plate with the other hand. It took approximately one minute to add all the PEG in one full rotation of the plate. Next, 8 ml DMEM was added down the edge of the plate while rotating the plate slowly. The PEG/DMEM mixture was aspirated gently from the cells and then 8 ml DMEM was used to rinse the cells. This DMEM was removed and 10 ml fresh DMEM was added and the cells were incubated for 30 min. at 37° C. Again the DMEM was aspirated from the cells and 10 ml DMEM in which 10% FBCS and 1×Pen/Strep were added, was added to the cells, which were then allowed to incubate overnight.

After incubation, the media was aspirated and the cells were rinsed with PBS. The cells were then trypsinized and divided among plates containing selection media (DMEM in which 10% FBS+1×Pen/Strep+1×HAT were added) so that each plate received approximately 100,000 cells. The media was changed on the third day following plating. Colonies were picked and placed into 24-well plates upon becoming visible to the naked eye (day 9-14). If a picked colony was confluent within 5 days, it was deemed healthy and the cells were trypsinized and moved to a 6-well plate.

DNA and stock hybrid cell cultures were prepared from the cells from the 6-well plate cultures. The cells were trypsinized and divided between a 100 mm plate containing 10 ml selection media and an Eppendorf tube. The cells in the tube were pelleted, resuspended 200 μl PBX and DNA was isolated using a Qiagen DNA mini kit at a concentration of <5 million cells per spin column. The 100 mm plate was grown to confluence, and the cells were either continued in culture or frozen.

### EXAMPLE 2

### Selecting Haploid Hybrids

Scoring for the presence, absence and diploid/haploid state of human chromosomes in each hybrid was performed using the Affymetrix, HuSNP genechip (Affymetrix, Inc,. of Santa Clara, Calif., HuSNP Mapping Assay, reagent kit and user manual, Affymetrix Part No. 900194), which can score 1494 markers in a single chip hybridization. As controls, the hamster and human diploid lymphoblastoid cell lines were screened using the HuSNP chip hybridization assay. Any SNPs which were heterozygous in the parent lymphoblastoid diploid cell line were scored for haploidy in each fusion cell line. Assume that “A” and “B” are alternative variants at each SNP location. By comparing the markers that were present as “AB” heterozygous in the parent diploid cell line to the same markers present as “A” or “B” (hemizygous) in the hybrids, the human DNA strands which were in the haploid state in each hybrid line was determined.

### EXAMPLE 3

### Long Range PCR

DNA from the hamster/human cell hybrids was used to perform long-range PCR assays. Long range PCR assays are known generally in the art and have been described, for example, in the standard long range PCR protocol from the Boehringer Mannheim Expand Long Range PCR Kit, incorporated herein by reference or all purposes.

Primers used for the amplification reactions were designed in the following way: a given sequence, for example the 23 megabase contig on chromosome 21, was entered into a software program known in the art herein called “repeat masker” which recognizes sequences that are repeated in the genome (e.g., Alu and Line elements)(see, A. F. A. Smit and P. Green, www.genome.washington.edu/uwgc/analysistools/repeatmask, incorporated herein by reference). The repeated sequences were “masked” by the program by substituting each specific nucleotide of the repeated sequence (A, T, G or C) with “N”. The sequence output after this repeat mask substitution was then fed into a commercially available primer design program (Oligo 6.23) to select primers that were greater than 30 nucleotides in length and had melting temperatures of over 65° C. The designed primer output from Oligo 6.23 was then fed into a program which then “chose” primer pairs which would PCR amplify a given region of the genome but have minimal overlap with the adjacent PCR products. The success rate for long range PCR using commercially available protocols and this primer design was at least 80%, and greater than 95% success was achieved on some portions of human chromosomes.

An illustrative protocol for long range PCR uses the Expand Long Template PCR System from Boehringer Mannheim Cat.# 1681 834, 1681 842, or 1759 060. In the procedure each 50 μL PCR reaction requires two master mixes. In a specific example, Master Mix 1 was prepared for each reaction in 1.5 ml microfuge tubes on ice and includes a final volume of 19 μL of Molecular Biology Grade Water (Bio Whittaker, Cat.# 16-001Y); 2.5 μL 10 mM dNTP set containing dATP, dCTP, dGTP, and dTTP at 10 mM each (Life Technologies Cat.# 10297-018) for a final concentration of 400 μM of each dNTP; and 50 ng DNA template.

Master Mix 2 for all reactions was prepared and kept on ice. For each PCR reaction Master Mix 2 includes a final volume of 25 μL of Molecular Biology Grade Water (Bio Whittaker); 5 μL 10×PCR buffer 3 containing 22.50 mM MgCl₂ (Sigma, Cat.# M 10289); 2.5 μL 10 mM MgCl₂ (for a final MgCl₂ concentration of 2.75 mM); and 0.75 μL enzyme mix (added last)

Six microliters of premixed primers (containing 2.5 μL of Master Mix 1) were added to appropriate tubes, then 25 μL of Master Mix 2 was added to each tube. The tubes were capped, mixed, centrifuged briefly and returned to ice. At this point, the PCR cycling was begun according to the following program: step 1: 94° C. for 3 min to denature template; step 2: 94° C. for 30 sec; step 3: annealing for 30 sec at a temperature appropriate for the primers used; step 4: elongation at 68° C. for 1 min/kb of product; step 5: repetition of steps 2-4 38 times for a total of 39 cycles; step 6: 94° C. for 30 sec; step 7: annealing for 30 sec; step 8: elongation at 68° C. for 1 min/kb of product plus 5 additional minutes; and step 9: hold at 4° C. Alternatively, a two-step PCR would be performed: step 1: 94° C. for 3 min to denature template; step 2: 94° C. for 30 sec; step 3: annealing and elongation at 68° C. for 1 min/kb of product; step 4: repetition of steps 2-3 38 times for a total of 39 cycles; step 5:94° C. for 30 sec; step 6: annealing and elongation at 68° C. for 1 min/kb of product plus 5 additional minutes; and step 7: hold at 4° C.

### EXAMPLE 4

### Wafer Design, Manufacture, Hybridization and Scanning

The set of oligonucleotide probes to be contained on an oligonucleotide array (chip or wafer) was defined based on the human DNA strand sequence to be queried. The oligonucleotide sequences were based on consensus sequences reported in publicly available databases. Once the probe sequences were defined, computer algorithms were used to design photolithographic masks for use in manufacturing the probe-containing arrays. Arrays were manufactured by a light-directed chemical synthesis processes which combines solid-phase chemical synthesis with photolithographic fabrication techniques. See, for example, WO 92/10092, or U.S. Pat. Nos. 5,143,854; 5,384,261; 5,405,783; 5,412,087; 5,424,186; 5,445,934; 5,744,305; 5,800,992; 6,040,138; 6,040,193, all of which are incorporated herein by reference in their entireties for all purposes. Using a series of photolithographic masks to define exposure sites on the glass substrate (wafer) followed by specific chemical synthesis steps, the process constructed high-density areas of oligonucleotide probes on the array, with each probe in a predefined position. Multiple probe regions were synthesized simultaneously and in parallel.

The synthesis process involved selectively illuminating a photo-protected glass substrate by passing light through a photolithographic mask wherein chemical groups in unprotected areas were activated by the light. The selectively-activated substrate wafers were then incubated with a chosen nucleoside, and chemical coupling occurred at the activated positions on the wafer. Once coupling took place, a new mask pattern was applied and the coupling step was repeated with another chosen nucleoside. This process was repeated until the desired set of probes was obtained. In one specific example, 25-mer oligonucleotide probes were used, where the thirteenth base was the base to be queried. Four probes were used to interrogate each nucleotide present in each sequence—one probe complementary to the sequence and three mismatch probes identical to the complementary probe except for the thirteenth base. In some cases, at least 10×10⁶ probes were present on each array.

Once fabricated, the arrays were hybridized to the products from the long range PCR reactions performed on the hamster-human cell hybrids. The samples to be analyzed were labeled and incubated with the arrays to allow hybridization of the sample to the probes on the wafer.

After hybridization, the array was inserted into a confocal, high performance scanner, where patterns of hybridization were detected. The hybridization data were collected as light emitted from fluorescent reporter groups already incorporated into the PCR products of the sample, which was bound to the probes. Sequences present in the sample that are complimentary to probes on the wafer hybridized to the wafer more strongly and produced stronger signals than those sequences that had mismatches. Since the sequence and position of each probe on the array was known, by complementarity, the identity of the variation in the sample nucleic acid applied to the probe array was identified. Scanners and scanning techniques used in the present invention are known to those skilled in the art and are disclosed in, e.g., U.S. Pat. No. 5,981,956 drawn to microarray chips, U.S. Pat. No. 6,262,838 and U.S. Pat. No. 5,459,325. In addition, U.S. Ser. No. 09/922,492, filed on Aug. 3, 2001, assigned to the assignee of the present invention, drawn to scanners and techniques for whole wafer scanning, is also incorporated herein by reference in their entireties for all purposes.

It will be understood by those of skill in the art that any suitable instrumentation or technology for the analysis of genetic information may be used to achieve the object of the invention.

### EXAMPLE 5

### Determination of SNP Haplotypes on Human Chromosome 21

Twenty independent copies of chromosome 21, representing African, Asian, and Caucasian chromosomes were analyzed for SNP discovery and haplotype structure. Two copies of chromosome 21 from each individual were physically separated using a rodent-human somatic cell hybrid technique, discussed supra. The reference sequence for the analysis consisted of human chromosome 21 genomic DNA sequence consisting of 32,397,439 bases. This reference sequence was masked for repetitive sequences and the resulting 21,676,868 bases (67%) of unique sequence were assayed for variation with high density oligonucleotide arrays. Eight unique oligonucleotides, each 25 bases in length, were used to interrogate each of the unique sample chromosome 21 bases, for a total of 1.7×10⁸ different oligonucleotides. These oligonucleotides were distributed over a total of eight different wafer designs using a previously described tiling strategy (Chee, et al., Science 274:610 (1996)). Light-directed chemical synthesis of oligonucleotides was carried out on 5 inch×5 inch glass wafers purchased from Affymetrix, Inc. (Santa Clara, Calif.).

Unique oligonucleotides were designed to generate 3253 minimally overlapping longe range PCR (LRPCR) products of 10 kb average length spanning 32.4 Mb of contiguous chromosome 21 DNA, and were prepared as described supra. PCR products corresponding to the bases present on a single wafer were pooled and hybridized to the wafer as a single reaction. In total, 3.4×10⁹ oligonucleotides were synthesized on 160 wafers to scan 20 independent copies of human chromosome 21 for DNA sequence variation.

SNPs were detected as altered hybridization by using a pattern recognition algorithm. A combination of previously described algorithms (Wang, et al., Science 280:1077 (1998)), was used to detect SNPs based on altered hybridization patterns. In total, 35,989 SNPs were identified in the sample of twenty chromosomes. The position and sequence of these human polymorphisms have been deposited in GenBank's SNPdb. Dideoxy sequencing was used to assess a random sample of 227 of these SNPs in the original DNA samples, confirming 220 (97%) of the SNPs assayed. In order to achieve this low rate of 3% false positive SNPs, stringent thresholds were required for SNP detection on wafers that resulted in a high false negative rate.

Over all, 47% of the 53,000 common SNPs with an allele frequency of 10% or greater estimated to be present in 32.4 Mb of the human genome were identified. This compares with an estimate of 18-20% of all such common SNPs present in the collection generated by the International SNP Mapping Working Group and the SNP Consortium. The difference in coverage is explained by the fact that the present study used larger numbers of chromosomes for SNP discovery. To assess the replicability of the findings, SNP discovery was performed for one wafer design with nineteen additional copies of chromosome 21 derived from the same diversity panel as the original set of samples. A total of 7188 SNPs were identified using the two sets of samples. On average, 66% of all SNPs found in one set of samples were discovered in the second set, consistent with previous findings (Marth, et al., Nature Genet. 27:371 (2001) and Yang, et al., Nature Genet. 26:13 (2000)). As expected, failure of a SNP to replicate in a second set of samples is strongly dependent on allele frequency. It was found that 80% of SNPs with a minor allele present two or more times in a set of samples were also found in a second set of samples, while only 32% of SNPs with a minor allele present a single time were found in a second set of samples. These findings suggest that the 24,047 SNPs in the collection with a minor allele represented more than once are highly replicable in different global samples and that this set of SNPs is useful for defining common global haplotypes. In the course of SNP discovery, 339 SNPs which appeared to have more than two alleles were identified.

In addition to the replicability of SNPs in different samples, the distance between consecutive SNPs in a collection of SNPs is critical for defining meaningful haplotype structure. Haplotype blocks, which can be as short as several kb, may go unrecognized if the distance between consecutive SNPs in a collection is large relative to the size of the actual haplotype blocks. The collection of SNPs in this study was very evenly distributed across the chromosome, even though repeat sequences were not included in the SNP discovery process.

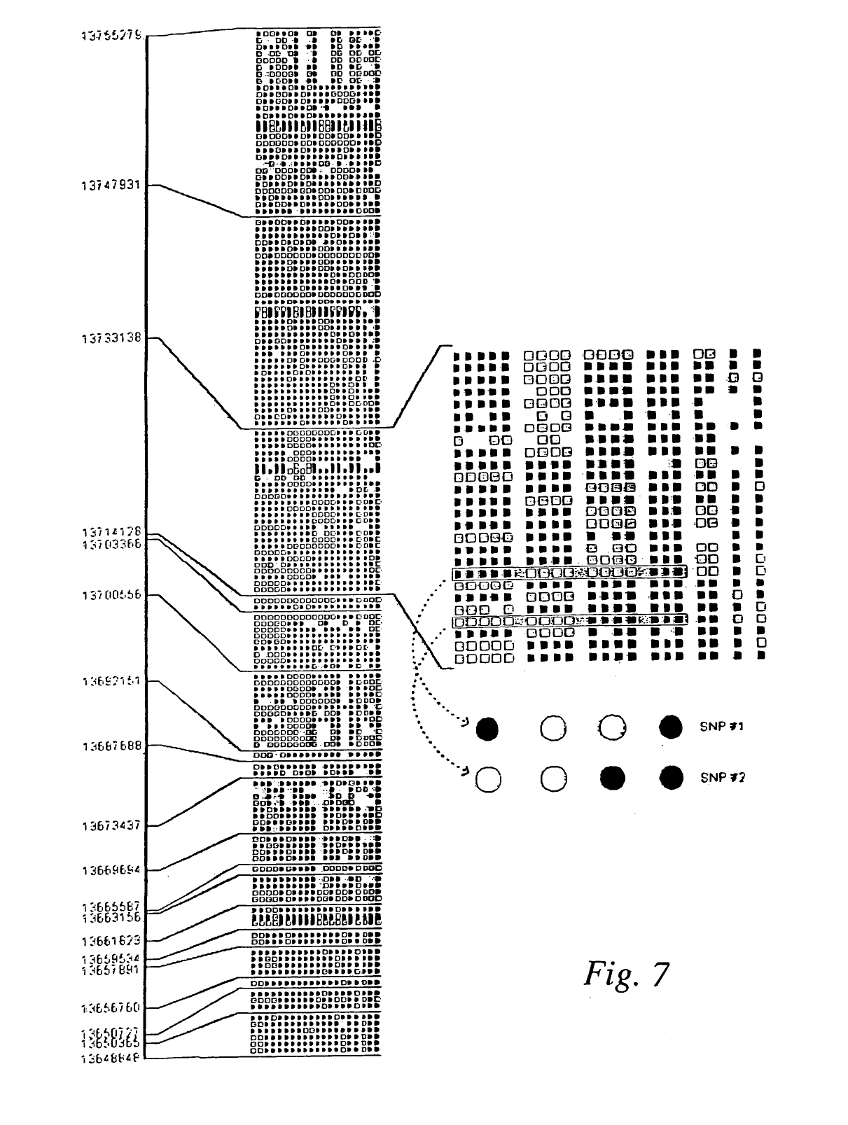

The present invention characterized SNPs on haploid copies of chromosome 21 isolated in rodent-human somatic cell hybrids were characterized, allowing direct determination of the full haplotypes of these chromosomes. The set of 24,047 SNPs with a minor allele represented more than once in the data set was used to define the haplotype structure are shown in FIG. 7. The haplotype patterns for twenty independent globally diverse chromosomes defined by 147 common human chromosome 21 SNPs is shown. The 147 SNPs span 106 kb of genomic DNA sequence. Each row of colored boxes represents a single SNP. The black boxes in each row represent the major allele for that SNP, and the white boxes represent the minor allele. Absence of a box at any position in a row indicates missing data. Each column of colored boxes represents a single chromosome, with the SNPs arranged in their physical order on the chromosome. Invariant bases between consecutive SNPs are not represented in the figure. The 147 SNPs are divided into eighteen blocks, defined by black horizontal lines. The position of the base in chromosome 21 genomic DNA sequence defining the beginning of one block and the end of the adjacent block is indicated by the numbers to the left of the vertical black line. The expanded boxes on the right of the figure represent a SNP block defined by 26 common SNPs spanning 19 kb of genomic DNA.

Of the seven different haplotype patterns represented in the sample, the four most common patterns include sixteen of the twenty chromosomes sampled (i.e. 80% of the sample). The black and white circles indicate the allele patterns of two informative SNPs, which unambiguously distinguish between the four common haplotypes in this block. Although no two chromosomes shared an identical haplotype pattern for these 147 SNPs, there are numerous regions in which multiple chromosomes shared a common pattern. One such region, defined by 26 SNPs spanning 19 kb, is expanded for more detailed analysis (see the enlarged region of FIG. 7). This block defines seven unique haplotype patterns in 20 chromosomes. Despite the fact that some data is missing due to failure to pass the threshold for data quality, in all cases a given chromosome can be assigned unambiguously to one of the seven haplotypes by the methods described herein. The four most frequent haplotypes, each of which is represented by three or more chromosomes, account for 80% of all chromosomes in the sample. Only two “informative” SNPs out of the total of twenty-six are required to distinguish the four most frequent haplotypes from one another. In this example, four chromosomes with infrequent haplotypes would be incorrectly classified as common haplotypes by using information from only these two informative SNPs. Several different possibilities exist in which three informative SNPs can be chosen so that each of the four common haplotypes is defined uniquely by a single SNP. One of these “three SNP” choices would be preferred over the two SNP combination in an experiment involving genotyping of pooled samples, since the two SNP combination would not permit determination of frequencies of the four common haplotypes in such a situation; thus, the present invention provides a dramatic improvement over the random selection method of SNP mapping.

The present invention allows one to define a set of contiguous blocks of SNPs spanning the entire 32.4 Mb of chromosome 21 while minimizing the total number of SNPs required to define the haplotype structure. In this experiment, an optimization algorithm based on a “greedy” strategy was used to address this problem. All possible blocks of physically consecutive SNPs of size one SNP or larger were considered. Considering the remaining overlapping blocks simultaneously, the block with the maximum ratio of total SNPs in the block to the minimal number of SNPs required to uniquely discriminate haplotypes represented more than once in the block was selected as described previously herein. Any of the remaining blocks that physically overlapped with the selected block were discarded, and the process was repeated until a set of contiguous, non-overlapping blocks that cover the 32.4 Mb of chromosome 21 with no gaps, and with every SNP assigned to a block, was selected. Given the sample size of twenty chromosomes, the algorithm produces a maximum of ten common haplotype patterns per block, with each pattern represented by two independent chromosomes.

Applying this algorithm to the data set of 24,047 common SNPs, 4135 blocks of SNPs spanning chromosome 21 were defined. A total of 589 blocks, comprising 14% of all blocks, contain greater than ten SNPs per block and include 44% of the total 32.4 Mb. In contrast, 2138 blocks, comprising 52% of all blocks, contain less than three SNPs per block and make up only 20% of the physical length of the chromosome. The largest block contains 114 common SNPs and spans 115 kb of genomic DNA. Overall, the average physical size of a block is 7.8 kb. The size of a block is not correlated with its order on the chromosome, and large blocks are interspersed with small blocks along the length of the chromosome.

Although the length of genomic regions with a simple haplotype structure is extremely variable, a dense set of common SNPs enables the systematic approach to define blocks of the human genome in which 80% of the global human population is described by only three common haplotypes. In general, when applying the particular algorithm used in this embodiment, the most common haplotype in any block is found in 50% of individuals, the second most common in 25% of individuals, and the third most common in 12.5% of individuals. It is important to note that blocks are defined based on their genetic information content and not on knowledge of how this information originated or why it exists. As such, blocks do not have absolute boundaries, and may be defined in different ways, depending on the specific application. The algorithm in this embodiment provides only one of many possible approaches. The results indicate that a very dense set of SNPs is required to capture all the common haplotype information. Once in hand, however, this information can be used to identify much smaller subsets of SNPs useful for comprehensive whole-genome association studies.

Those skilled in the art will appreciate readily that the techniques applied to human chromosome 21 can be applied to all the chromosomes present in the human genome. In fact, multiple whole genomes of a diverse population representative of the human species may be used to identify SNP haplotype blocks common to all or most members of the species.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

The present invention provides greatly improved methods for conducting genome-wide association studies by identifying individual variations, determining SNP haplotype blocks, determining haplotype patterns and, further, using the SNP haplotype patterns to identify informative SNPs. It is to be understood that the above description is intended to be illustrative and not restrictive. Many embodiments will be apparent to those skilled in the art upon reviewing the above description. The scope of the invention should, therefore, be determined not with reference to the above description, but should instead be determined with reference to the appended claims, along with the full scope of equivalents to which such claims are entitled.

What is claimed is:

1. A business method comprising:
identifying disease related loci by comparing frequencies of SNP haplotype patterns from essentially coding regions of individuals in a control group with frequencies of SNP haplotype patterns from essentially coding regions of individuals in a disease group, wherein differences in said frequencies indicate locations of disease-related genetic loci;
using said disease related loci in a discovery process; and
collaboratively or independently, marketing products from said discovery process.

2. The method of claim 1, wherein said control group comprises at least 16 individuals.

3. The method of claim 1, wherein said disease group comprises at least 16 individuals.

4. The method of claim 1, wherein said SNP haplotype patterns are determined using informative SNPs.

5. A business method comprising:
a. making an association between SNP haplotype patterns from coding regions of a genome and a phenotypic trait of interest by
i. building a baseline of SNP haplotype patterns from regions consisting essentially of coding regions of a genome;
ii. pooling genomic DNA from a population having a common phenotypic trait of interest;
iii. identifying said SNP haplotype patterns that are associated with said phenotypic trait of interest;
b. using said association in a discovery process; and
c. collaboratively or independently, marketing products from said discovery process.

6. The method of claim 5, wherein the phenotypic trait of interest is a drug response state.

7. The method of claim 6, wherein the drug response state is a responder state.

8. The method of claim 6, wherein the drug response state is a toxicity state.

9. The method of claim I or 5 wherein the step of collaboratively or independently marketing products comprises receiving funds from a partner for making the association between the SNP patterns and the phenotypic trait.

10. The method of claim 5, wherein a technology provider provides discounted technology for said association, and receives equity in return for said discounted technology.

11. The method of claim 5, wherein said discovery process comprises identifying a pharmaceutical compound to address said phenotypic trait.

12. The method of claim 11, wherein said pharmaceutical compound is an antisense compound.

13. The method of claim 11, wherein said pharmaceutical compound is a small organic molecule.

14. The method of claim 11, wherein said pharmaceutical compound is an antibody.

15. The method of claim 11, wherein said pharmaceutical compound is a protein compound.
